(19) United States
(12) Patent Application Publication
(10) Pub. No.: US 2012/0041205 A1
(43) Pub. Date: Feb. 16, 2012

(54) PREPARATION OF DIHYDROPYRROL DERIVATIVES AS INTERMEDIATES

(76) Inventors: Christophe Pfleger, Riedisheim (FR); Paul Spurr, Riehen (CH); Rene Trussardi, Birsfelden (CH); Pius Waldmeier, Wegenstetten (CH); Shaoning Wang, Basel (CH)

(21) Appl. No.: 13/278,718

(22) Filed: Oct. 21, 2011

Related U.S. Application Data
(62) Division of application No. 12/313,988, filed on Nov. 26, 2008, now abandoned.

(30) Foreign Application Priority Data
Nov. 29, 2007 (EP) 07121841.6

(51) Int. Cl. C07D 471/04; C07D 209/44

(52) U.S. Cl. 546/113; 548/482

(57) ABSTRACT

The invention is concerned with a new scalable process for the preparation of compounds of formula I comprising a new process for the preparation of the key intermediate, a dihydropyrrole derivative formula II or a salt thereof.

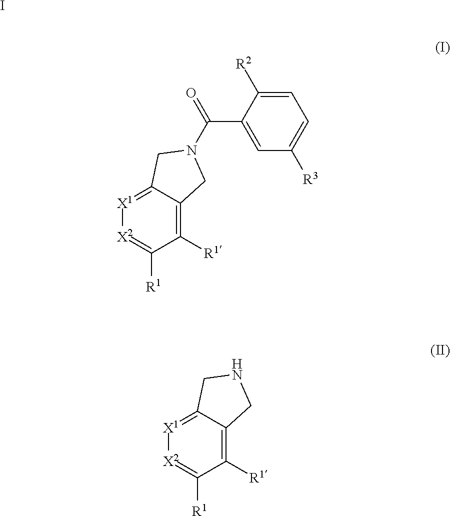

### PRIORITY TO RELATED APPLICATIONS

This application is a division of U.S. application Ser. No. 12/313,988, filed Nov. 26, 2008, now pending; which claims the benefit of European Application No. 07121841.6 filed Nov. 29, 2007. The entire contents of the above-identified applications are hereby incorporated by reference.

### FIELD OF THE INVENTION

The invention is concerned with a new scalable process for the preparation of compounds of formula I or II which are glycine transport inhibitors or are used in the preparation of pharmaceutical active ingredients which process is amenable to large scale manufacture.

### BACKGROUND OF THE INVENTION

Compounds of formula I as described herein are glycine transporter inhibitors and are suitable for the treatment of neurological and neuropsychiatric disorders. The majority of diseases states implicated are psychoses, schizophrenia (Armer R E and Miller D J, 2001, Exp. Opin. Ther. Patents, 11 (4): 563-572), psychotic mood disorders such as severe major depressive disorder, mood disorders associated with psychotic disorders such as acute mania or depression associated with bipolar disorders and mood disorders associated with schizophrenia, (Pralong E. T. et al., 2002, Prog. Neurobiol., 67: 173-202), autistic disorders (Carlsson M. L., 1998, J. Neural Transm. 105: 525-535), cognitive disorders such as dementias, including age related dementia and senile dementia of the Alzheimer type, memory disorders in a mammal, including a human, attention deficit disorders and pain (Armer R E and Miller D J, 2001, Exp. Opin. Ther. Patents, 11 (4): 563-572).

A method for the preparation of compounds of formula I is described in WO 2006/082001. This method comprises 9 steps and is not suitable for large-scale production. It has several drawbacks such as the price and the availability of the starting materials; it involves problematic steps such as a Sandmeyer reaction, a low overall yielding reduction step as well as chromatographic purifications of the intermediates. \

Compounds of formula II are useful intermediates for the preparation of HCV protease inhibitors as disclosed in WO2005/037214 and WO2005/095403.

### SUMMARY OF THE INVENTION

The present invention provides a process for the manufacture of a compound according to formula I or II which are useful medicaments or useful intermediates for the preparation of medicaments.

### DETAILED DESCRIPTION OF THE INVENTION

The invention provides a new process for the preparation of compounds of formula I, especially for the preparation of compounds of formula Ia wherein:

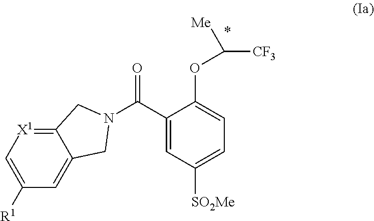

X¹ is N or C, preferably N;

R¹ is hydrogen, hydroxy, fluoro, chloro, lower alkyl optionally substituted by halogen or hydroxy, cycloalkyl, lower alkoxy optionally substituted by halogen, —CN, —NR⁷R^{7′}—S-lower alkyl, —S(O)₂-lower alkyl, —O—(CH₂)_{y}-lower alkoxy, —O(CH₂)_{y}C(O)N(lower alkyl)₂, —C(O)-lower alkyl, —C(O)O-lower alkyl, —C(O)—NH-lower alkyl, or —C(O)—N(lower alkyl)₂;

y is 1, 2, 3 or 4;

R⁷ and R^{7′} are independently hydrogen or lower alkyl; and

* denotes a chiral center.

According to the present invention compounds of formula I, and especially

compounds of formula Ia, wherein R¹ is trifluoromethyl can be prepared in high

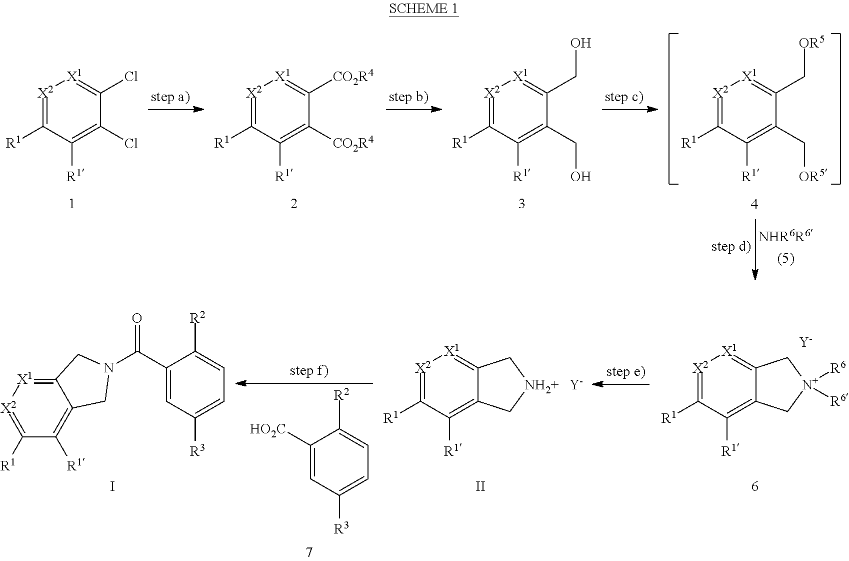

yields and a scalable manner as depicted in reaction SCHEME 1 wherein the symbols X¹, R¹ and R⁷ and R^{7′} and y are as defined above, R^{1′} is independently selected from groups provided for R¹ above; R² is halogen, lower alkyl optionally substituted by halogen or hydroxy, lower alkoxy optionally substituted by halogen, cycloalkyl, —NR⁷R^{7′}, cyclic amine, heterocycloalkyl, aryl or 5- or 6-membered heteroaryl, containing one, two or three heteroatoms, selected from the group consisting of oxygen, sulphur or nitrogen; R³ is —S(O)₂-lower alkyl, —S(O)₂NH-lower alkyl, —NO₂ or —CN;

R⁴ and R⁴′ are independently C₁-C₆-alkyl;

R⁵ and R⁵′ are independently a leaving group such as nosylate, tosylate, mesylate;

R⁶ and R⁶′ are independently of each other hydrogen, diphenylmethyl, benzyl, diallyl or allylbenzyl;

Y⁻ is an anion of an acid HY such as hydrochloric acid, hydrofluoric acid, hydrobromic acid, or hydriodic acid, methane sulfonic acid, toluene sulfonic acid or 4-nitrotoluene sulfanic acid.

Compounds of formula 7 are available commercially or can be prepared according to methods described in the art, for example in WO 2006/082001.

Compound of formula 7, wherein R² is —O—CH(CH₃)—CF₃ and R³ is —SO₂CH₃ can be prepared by enzymatic reduction of 1,1,1-trifluoro-propan-2-one with Baker's Yeast to yield the optically active (S)-1,1,1-trifluoro-propan-2-ol, which is then reacted with a fluoro substituted aryl group in the presence of a base and in an appropriate solvent as described in EP application Ser. No. 07/103,485.4 filed on Mar. 5, 2007.

Alternatively, (S)-1,1,1-trifluoro-propan-2-ol can be prepared via a conventional enzymatic racemate resolution approach and further reacted as described in 07/103,485.4 filed on Mar. 5, 2007.

Yet another method to prepare compound 7 wherein R² is —O—CH(CH₃)—CF₃ and R³ is —SO₂CH₃, is by asymmetric hydrogenation of 1,1,1-trifluoro-propan-2-one and subsequent coupling with 2-fluoro-5-methane sulfonyl-benzoic acid. The asymmetric hydrogenation of 1,1,1-trifluoro-propan-2-one can be carried out with a Ru biphosphine complex such as (S)-3,5-tent-pentyl-MeOBIPHEP (Phosphine, (6,6′-dimethoxy[1,1′-biphenyl]-2,2′-diyl)bis[bis(3,5-di-tert-pentyl-phenyl)-) or (S)-3,5-tert-butyl-MeOBIPHEP (Phosphine, (6,6′-dimethoxy[1,1′-biphenyl]-2,2′-diyl)bis[bis(3,5-di-tert-butyl-phenyl)-) as described in EP 06/117928.9 filed on Jul. 27, 2006.

The process according to the invention is a 6 step synthesis of compounds of formula I, especially of a compound of formula Ia. The key intermediate of formula II is prepared in a 4 step synthesis according to the invention and coupled to a compound of formula 7. The reaction steps of this synthesis are described in more detail below:

step a) Carbonylation of a compound of formula 1 to afford the diester of formula 2 as depicted in step a of SCHEME 1 wherein R¹, R¹′, R⁴, R^{4′}, X¹ and X² are defined as above.

In one embodiment R⁴ and R^{4′} are independently of each other lower alkyl, R^{1′} is hydrogen and X² is carbon (C).

The double alkoxycarbonylation of a compound of formula 1 to afford the dialkyl ester 2 is carried out with CO in the presence of a catalyst such as PdCl₂(dppf)CH₂Cl₂ (dppf being 1,1′-bis(diphenylphosphino)ferrocene, PdCl₂(dppf), PdCl₂(PPh₃)₂, (PPh₃ being triphenylphosphine) Pd(OAc)₂(PPh₃)₂(Pd(OAc)₂ being palladium diacetate), Pd(OAc)₂(dppf), preferred is PdCl₂(dppf)CH₂Cl₂ and in the presence of a base such as triethylamine, Na₂CO₃, preferred is sodium acetate optionally water free in a lower alcohol such as methanol or ethanol. The reaction temperature is 80 to 150° C., preferred is 120° C., the pressure from 5 to 100 bar, preferably 15 bar, the reaction time is from 2.5 to 24 h, preferably 18 h.

In one embodiment the catalyst is used as a complex (0.1-0.3 mol-%) rather than being prepared in situ, where the amount of metal precursor is 0.5 mol-% Pd(OAc)₂ and the ligand dppf is 3 mol-%.

For compounds of formula I, wherein X¹ and X² are C, step a) can be omitted and the readily available substituted phthalic acid derivatives can be used as starting materials in step b).

Step b) Reduction of the diester 2 to the diol 3 as depicted in step b of SCHEME 1 wherein R¹, R¹′, X¹ and X² are defined as above and preferably wherein R^{1′} is hydrogen X² is C and X¹ is N and R¹ is a s defined above.

The reduction of 2 can be carried out in the presence of reduction reagent such as NaBH₄ in acetic acid, DIBAH (diisobutyl aluminum hydride),), REDAL (bis-(2-methoxyethoxy)aluminium hydride), LiBH₄ or with LiAlH₄, and optionally an alkali or earth alkali salts such as BaCl₂, CaCl₂, MgCl₂, MnCl₂, FeCl₂, YCl₃, CeCl₃, SrCl₂, ZnCl₂, ZrCl₄, LiCl.MgCl₂ and/or CaCl₂, especially CaCl₂.

In one embodiment of the invention however, reduction of a diester of formula 2 wherein R¹ is trihaloalkyl and R¹′ is hydrogen is carried out in the presence of a reduction agent as NaBH₄ in acetic acid, DIBAH, LiBH₄ or LiAlH₄ and an alkali or earth alkali salts such as BaCl₂, CaCl₂, MgCl₂, MnCl₂, FeCl₂, YCl₃, CDCl₃, SrCl₂, ZnCl₂, ZrCl₄, LiCl.MgCl₂ and/or CaCl₂. The reaction is carried out in solvents such as a lower alcohol, for example in methanol (MeOH) or ethanol (EtOH) or in a mixture of a lower alcohol and an ether, for example in (EtOH)/tetrahydrofuran (THF).

In one embodiment of the invention the reduction of compounds of formula 2, wherein R¹ is trihaloalkyl and R¹′ is hydrogen, is carried out with NaBH₄ in the presence of MgCl₂ in ethanol or ethanol/tetrahydrofuran at a temperature of 0° C. to 50° C., preferably at a temperature of 25-30° C. Work-up of the reaction mixture is carried out by addition of a base such as aqueous bicarbonate, elimination of the solvent, followed by extraction with an acid such HCl or citric acid. Formation of the corresponding boric acid ester is avoided for example by complete removal of the solvent, for example EtOH and by adjusting the pH to >7.

Step c) Activation of diol 3 by converting the alcohols to a more efficient leaving group as depicted in step c of SCHEME 1 wherein R¹, R¹′, R⁵, R⁵′, X¹ and X² are as defined above, preferably wherein R^{1′} is hydrogen X² is C and X¹ is N and R¹ is as defined above.

The diol derivative 4 is formed according to methods known in the art, for example in a solvent wherein the salts formed during the reaction are insoluble and easily removable by filtration. The reaction can be carried in presence of a base such diisopropylethylamine (DIPEA), triethylamine (NEt₃) in polar solvents such as EtOAc at temperatures from −20 to 40° C. For example the reaction can be carried out using mesyl chloride and NEt₃ in EtOAc at a temperature of about 0° C. The resulting salts are filtered off and the filtrate containing the compound of formula 4 can be used in the cyclization step d) without further purification. Complete conversion of the diol to its derivative 4 can be reached from anhydrous diol.

Step d) is an cyclization of a compound of formula 4 with an amino derivative as depicted in step d of SCHEME 1 wherein R¹, R¹′, R⁵, R⁵′, X¹ and X² are as defined herein above, R⁶, R⁶′ are independently of each other hydrogen, an amino protecting groups such as benzyl, diphenylmethyl or the like; and Y⁻ is anion such as Cl⁻, F⁻, Br⁻, I⁻, mesylate, tosylate or nosylate, preferably wherein R^{1′} is hydrogen X² is C and X¹ is N and R¹ is a s defined above. For compounds of formula 6, wherein R⁶′ is hydrogen, the free acid or the corresponding salt can be isolated.

In one embodiment the amino derivative 5 is diphenylmethyl amine (R⁶ is diphenylmethyl and R⁶′ is hydrogen). The cyclization to form the diphenylmethyl derivative 6 the reaction is carried out at a temperature of 60° C. to 100° C. in a solvent such as THF, MeOH or dimethylformamide (DMF) or mixtures thereof, in the presence of a base such as DIPEA, K₂CO₃ or NEt₃.

In another embodiment the amino derivative 5 is dibenzyl amine (R⁶ and R⁶′ are benzyl). For the preparation of the dibenzylamino derivative 6 the reaction can be carried out with dibenzylamine and in the presence of a base such as DIPEA in THF, MeOH or DMF preferably in EtOAc preferably under reflux conditions. The pure product precipitated directly during the reaction as the corresponding salt and is stable against oxidation on air.

The purification of the product of formula 6 can be accomplished by crystallization and/or formation of its salt. In one embodiment the hydrochloride salt of formula 6 is formed using acetyl chloride in MeOH. In another embodiment the compound of formula 6 can be isolated directly from the reaction mixture as its mesylate, tosylate or nosylate salt.

Step e) is the hydrogenolysis of the protected amine 6 or a salt thereof, to afford a compound of formula II, or a salt thereof, wherein R¹, R¹′, X¹ and X² are as defined above, preferably wherein R^{1′} is hydrogen X² is C and X¹ is N and R¹ is a s defined above.

The hydrogenolysis is accomplished with hydrogen using catalytic amounts of Pd/C in a suitable solvent. Suitable solvents are for example alcohols, such as MeOH, EtOH and the like or, ethers such as THF, or hydrocarbons such as toluene, or mixtures thereof. Preferably the reaction is carried out at RT.

In an embodiment an acid such as for example hydrochloric acid is used during the hydrogenolysis and the corresponding salt of compound II is obtained.

In a further embodiment a salt, preferably the mesylate or hydrochloride salt of the compound of formula II is prepared by hydrogenation of the corresponding salt of formula 6 in the presence of a catalyst such as Pd/C (10%) in an alcohol under a hydrogen atmosphere. The reaction is carried out at a temperature of 0 to 50° C.

In a further embodiment the hydrogenation of a compound of formula 6 wherein R¹ is trihaloalkyl and R¹′ is hydrogen is carried out under 1 atmosphere of hydrogen, in a suitable solvent such as an alcohol, for example in methanol. About 1 to 10%, preferably about 2.5% catalyst such as Pd/C or Pd(OH)₂/C is used.

Step f) is acylation of a compound of formula II, or a salt thereof, by a suitable acid of formula 7 in the presence of an activating agent such as POCl₃, (COCl)₂, SOCl₂, or the like, or any peptide coupling reagents like HATU, TBTU, CDI, to afford the desired compound of formula I.

In an embodiment a salt, preferably the hydrochloride or the mesylate salt of the compound of formula II, wherein R¹′ is hydrogen and X² is carbon (C), is reacted with a compound of formula 7, wherein R² is alkoxy substituted by halogen.

In an embodiment of the present invention there is provided a process for the preparation of a compound of formula II as provided in steps a) through e) above.

As used herein the term “halogen” denotes fluorine, chlorine, iodine and bromine, preferably fluorine and iodine.

The term “lower alkyl” denotes a saturated straight- or branched-chain group containing from 1 to 6 carbon atoms, for example, methyl, ethyl, propyl, isopropyl, n-butyl, iso-butyl, 2-butyl, tert-butyl, pentyl, hexyl and the like. Preferred lower alkyl groups are groups with 1-4 carbon atoms.

The term “lower alkyl optionally substituted by halogen or hydroxy” denoted lower alkyl groups as defined above which group are unsubstituted or substituted by one or several substituents individually selected from halogen as defined above or hydroxy. Examples of substituted lower alkyl groups are trifluoromethyl (CF₃), difluoromethyl (CHF₂), fluoromethyl (CH₂F), 2,2,2-trifluoroethyl (—CH₂CF₃), 2,2-difluoroethyl (—CH₂CHF₂), 2-fluoroethyl (—CH₂CH₂F), 3,3,3-trifluoropropyl (—CH₂CH₂CF₃), 2,2-difluoropropyl (—CH₂—CF₂—CH₃), 4,4,4-trifluorobutyl (—CH₂CH₂CH₂CF₃), 1-trifluoromethyl-propyl CH(CF₃)CH₂CH₃,2,2,2-trifluoro-1,1-dimethyl-ethyl C[(CH₃)₂]-CF₃,2-chloroethyl (CH₂CH₂Cl, 2,2,3,3,3-pentafluoro-propyl (CH₂CF₂CF₃), 2,2,3,3-tetrafluoro-propyl (CH₂CF₂CHF₂), 2,2,2-trifluoro-1,1-dimethylethyl (C(CH₃)₂CF₃, CH(CH₃)CF₃) or 2-fluoro-1-fluoromethyl-ethyl (CH(CH₂F)CH₂F). Preferred are CH₂CF₃, CF₃ or CH(CH₃)CF₃.

The term “lower alkoxy” denotes a saturated straight- or branched-chain alkyl group containing from 1 to 6 carbon atoms as described above and which groups are connected via an oxygen atom.

The term “lower alkoxy, optionally substituted by halogen” denotes a lower alkyl group as defined above attached via an oxygen group, said lower alkyl group being unsubstituted or substituted by one or several substituents individually selected from halogen as defined above. Examples of such groups are trifluoromethoxy (—OCF₃), difluoromethoxy (—O—CHF₂), fluoromethoxy (—O—CH₂F), 2,2,2-trifluoroethoxy (—O—CH₂CF₃), 2,2-difluoroethoxy (—O—CH₂CHF₂), 2-fluoroethoxy (—O—CH₂CH₂F), 3,3,3-trifluoropropyloxy (—O—CH₂CH₂CF₃), 4,4,4-trifluorobutoxy (—O—CH₂CH₂CH₂CF₃), 1-trifluoromethyl-propyloxy (—O—CH(CF₃)CH₂CH₃), 2,2,2-trifluoro-1,1-dimethyl-ethoxy (—O—C[(CH₃)₂]-CF₃), 2-chloroethoxy (—O—CH₂CH₂Cl), 2,2,3,3,3-pentafluoro-propyloxy (—O—CH₂CF₂CF₃), 2,2,3,3-tetrafluoro-propyloxy (—O—CH₂CF₂CHF₂), 2,2,2-trifluoro-1,1-dimethyl-ethoxy (—O—C(CH₃)₂CF₃), 2,2,2-trifluoro-1-methyl-ethoxy (—O—CH(CH₃)CF₃) or 2-fluoro-1-fluoromethyl-ethoxy (—O—CH(CH₂F)CH₂F). Preferred are —O—CH₂CF₃, —O—CF₃ or —O—CH(CH₃)CF₃.

The term “cycloalkyl” denotes a cyclic alkyl group having 3 to 8 carbon atoms such as cyclopropyl, cyclobutyl, cyclopentyl, cyclohexyl, cycloheptyl or cyclooctyl.

The term “cyclic amine” denotes a saturated carbon ring, containing from 3 to 6 carbon atoms as defined above, and wherein at least one of the carbon atoms is replaced by a heteroatom, selected from the group consisting of N, O or S and wherein the N-atom is linked to the phenyl ring, for example piperidine, piperazine, morpholine, thiomorpholine, di-oxo-thiomorpholine, pyrrolidine, pyrazoline, imidazolidine, azetidine, and the like. Such groups can be substituted by one or more substituents, selected from the group consisting of halogen, hydroxy, phenyl, lower alkyl, lower alkoxy or ═O.

As used herein, the term “heterocycloalkyl” denotes a saturated carbon ring, containing from 3 to 6 carbon atoms, and wherein at least one of the carbon atoms is replaced by a heteroatom, selected from the group consisting of N, O or S. Examples of such groups are tetrahydropyran-2, 3 or 4-yl, tetrahydrofuran-2 or 3-yl, oxetan-3-yl, [1,4]dioxin-2-yl and the like.

The term “aryl” denotes a mono- or bicyclic aromatic carbocycle, for example phenyl, benzyl or naphthyl.

The term “5 or 6-membered heteroaryl” denotes for example furanyl, thienyl, pyrrolyl, imidazolyl, pyrazolyl, triazolyl, thiazolyl, isothiazolyl, isoxazolyl, pyridinyl, pyrazinyl, pyrimidinyl or the like.

The term “pharmaceutically acceptable acid addition salts” embraces salts with inorganic and organic acids, such as hydrochloric acid, hydrobromic acid, hydrofluoric acid, hydriodic acid, nitric acid, sulfuric acid, phosphoric acid, citric acid, formic acid, fumaric acid, maleic acid, acetic acid, succinic acid, tartaric acid, methanesulfonic acid, p-toluenesulfonic acid and the like.

The term “leaving group” denotes groups such as Cl⁻, Br⁻, I⁻, nosyl⁻(3-nitrobenzenesulfonate, 3-NO₂—C₆H₄—SO₃⁻), mesyl⁻(methylsulfonyl, CH₃SO₃⁻) or tosyl⁻(p-toluenesulfonyl, 4-(CH₃)—C₆H₄—SO₃⁻).

In another embodiment of the present invention there is provided a process for the preparation of a compound according to formula I wherein R¹, R¹′, X¹, X², R² and R³ are as defined above, which process comprises the step of coupling a compound according to formula II with a carboxylic acid 7, in the presence of an activating agent such as POCl₃, (COCl)₂, SOCl₂, or the like, or any peptide coupling reagents like HATU, TBTU, CDI, to afford the desired compound of formula I.

In yet another embodiment of the present invention there is provided a process for the preparation of a compound according to formula Ia wherein R¹ and X¹ are as defined above, which process comprises the step of coupling a compound according to formula II with 5-methanesulfonyl-2-(2,2,2-trifluoro-1-methyl-ethoxy)-benzoic acid.

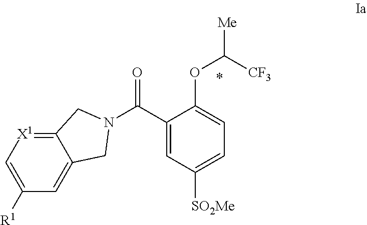

The following examples illustrate the processes claimed herein. These examples are provided to enable those skilled in the art to more clearly understand and to practice the present invention. They should not be considered as limiting the scope of the invention, but merely as being illustrative and representative thereof.

### EXAMPLES

### 1. Synthesis of 2,3-pyridine dicarboxylic acid-5-trifluoromethyl diethyl ester

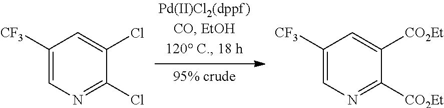

A 200 L autoclave was charged with a solution of 2,3-dichloro-5-trifluoromethyl pyridine (14.6 kg, 67.6 mol) in EtOH (60.0 L) and was treated at RT under argon with a suspension of Pd(II) Cl₂(dppf) (73 g, 89.4 mmol) and sodium acetate (13.6 kg, 165.8 mol) in 54 l ethanol. The autoclave was sealed, the carbon monoxide pressure raised to 15 bar and heated to 120° C. with stirring at for 18 h then cooled to 22° C. and vented.

The black suspension was filtered and the vessel rinsed twice with 50 L (100 L total) of EtOH. The filtrate was evaporated under reduced pressure at 50° C. and the residue dissolved in a mixture of EtOAc (30 L) and toluene (15 L). The solution was washed with water (30 L) and after phase separation the organic phase was evaporated under reduced pressure at 50° C. to yield 18.7 kg (95%) of 2,3-pyridine dicarboxylic acid-5-trifluoromethyl diethyl ester as an oil: MS (EI): 292 (M+H⁺, 17), 272 (M−F, 12), 246 (32), 218 (100), 191 (17), 174 (47), 147 (81).

### 2.1. Synthesis of (2-hydroxymethyl-5-trifluoromethyl-pyridin-3-yl)-methanol

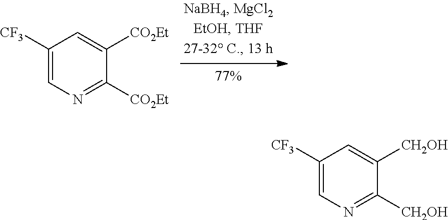

A suspension of MgCl₂ (1.63 kg, 16.8 mol) in THF (14.5 L) was treated at Tᵢₘₐₓ=10° C. over 35 min with EtOH (11.8 L) (exothermic reaction). The reaction mixture was vigorously stirred and treated in portions (over 30-60 min, temperature controlled) with a suspension of sodium borohydride (1.3 kg, 32.99 mol) in THF (6.7 L). A very exothermic reaction ensued, accompanied by foaming. The mixture was warmed to 28° C. and stirred at this temperature over 20 min. To the suspension was added over 35 min at Tᵢ=27-32° C. (Tᵢ=internal temperature) a solution of 2,3-pyridine dicarboxylic acid-5-trifluoromethyl diethyl ester (1.69 kg, 5.7 mol) in EtOH (5.1 L). The yellow-orange suspension was stirred for 13 h at Tᵢ=27-32° C.

The yellow reaction mixture was added in portions over 18 min to a solution of NaHCO₃ (5.9 kg, 81.9 mol) and H₂O (68 L) resulting in an exothermic reaction and gas evolution! The yellow suspension was stirred for 18 min, then all of the organic solvent was removed by evaporation under reduced pressure (400-50 mbar) at 60° C. The total volume was adjusted with water to 70 L. The suspension was treated under vigorous stirring with citric acid (10.5 kg, 54.3 mol) dissolved in H₂O (8.4 L) to adjust the pH to 7 (foaming). To the yellow solution was added 40 l tert-butyl methyl ether. After extraction and phase separation, the water phase was extracted twice with tert-butyl methyl ether (30 L, 60 Lin total). The combined organic phases were dried (15 kg Na₂SO₄), filtered and evaporated at 60° C. and 750-10 mbar to afford 913.0 g (76.9%) of (2-hydroxymethyl-5-trifluoromethyl-pyridin-3-yl)-methanol as a yellow-red oil: MS (EI): 208 (M+H⁺, 13), 289 (54), 161 (100).

### 2.2 Preparation of (2-Fluoro-6-hydroxymethyl-phenyl)-methanol starting from 3-Fluoro-phthalic acid dimethyl ester

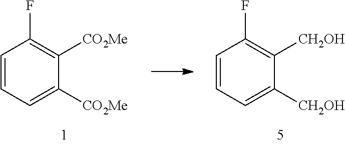

In a 10 mL two necked round bottom flask equipped with a thermometer, magnetic stirrer and an inert gas supply was charged with of sodium dihydrido-bis-(2-methoxyethoxy)aluminate (1.20 mL, 4.2 mmol, 3.5M solution in toluene) and cooled to 0-5° C. A solution of 3-fluorophthalic acid dimethylester (212.0 mg, 1.0 mmol) in THF (2.2 mL) was added dropwise over 5 min at 0-5° C. The reaction mixture was stirred for 1.5 h at 0-5° C. A solution of brine (1.05 mL) and 2M NaOH (1.05 mL) was added dropwise at 0-5° C. over 5 min. The reaction mixture was extracted with tert-butyl methyl ether (3.0 mL), the organic phase was separated and dried (Na₂SO₄), filtered and evaporated at 40° C. and 190-9 mbar to afford 110.0 mg (70%) of (2-fluoro-6-hydroxymethyl-phenyl)-methanol as an off white solid: MS (EI): 138 (100), 137 (78), 109 (57).

### 2.3. Preparation of (2-fluoro-6-hydroxymethyl-phenyl)-methanol (5) starting from fluoro-phthalic acid

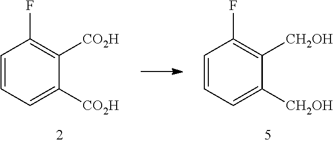

A 500 mL four necked round bottom flask equipped with a thermometer, mechanic stirrer and an inert gas supply was charged with 3-fluorophthalic acid (18.41 g, 100 mmol) dissolved in THF (92 mL) and cooled to 0-5° C. A solution of sodium dihydrido-bis(2-methoxyethoxy)aluminate (143 mL of a 3.5M solution) in toluene (500 mL) was added over 93 min. The reaction mixture was stirred for 1 h at 0-5° C. and then for 2 h at RT. The light orange solution was cooled to 0-5° C. and a solution of brine (125 mL) and 2M NaOH (125 mL) was added dropwise over 13 min. The organic phase was separated and the aqueous solution was extracted with tert-butyl methyl ether (240 mL), the combined organic phases were dried (280 g Na₂SO₄), filtered and evaporated in a rotary evaporator at 40° C. and 300-10 mbar. The residue was treated three times with a solution of tert-butyl methyl ether (10 mL) and toluene (50 mL) and evaporated at 40° C. and 300-10 mbar to afford 14.25 g (91%) of (2-fluoro-6-hydroxymethyl-phenyl)-methanol as beige crystals: MS (EI): 138 (100), 137 (79), 109 (57).

### 2.4. Preparation of (2-fluoro-6-hydroxymethyl-phenyl)-methanol (5) starting from fluoro phthalic acid anhydride

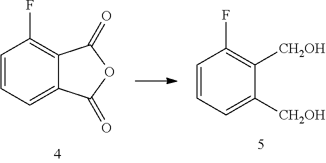

A 10 mL two necked round bottom flask equipped with a thermometer, magnetic stirrer and an inert gas supply was charged with 3.5M solution of sodium dihydrido-bis(2-methoxyethoxy)aluminate in toluene (1.20 mL, 4.2 mmol) and cooled to 0-5° C. A solution of 4-fluoro-isobenzofuran-1,3-dione (166.1 mg, 1.0 mmol) and THF (1.60 mL) was added dropwise over 5 min at 0-5° C. The reaction mixture was stirred for 4.5 h at 0-5° C. A solution of brine (1.05 mL) and 2M sodium hydroxide (1.05 mL) was added dropwise at 0-5° C. over 5 min. The reaction mixture was extracted with tert-butyl methyl ether (3.0 mL), the organic phase was separated and dried (Na₂SO₄), filtered and evaporated at 40° C. 190-9 mbar to afford 104.0 mg (68%) of (2-fluoro-6-hydroxymethyl-phenyl)-methanol as an off white solid: MS (EI): 138 (100), 137 (96), 109 (64).

### 2.5. Preparation of (2-fluoro-6-hydroxymethyl-phenyl)-methanol starting from fluoro phthalic acid mono ethyl ester

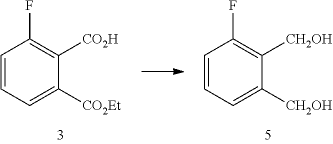

A 25 mL two necked round bottom flask equipped with a thermometer, magnetic stirrer and an inert gas supply was charged with a solution of 3-fluorophthalic acid 1-ethyl ester (424.4 mg, 2.0 mmol) and THF (2.12 mL) and cooled to 0-5° C. A solution of REDAL (2.86 mL, 10.0 mmol, 3.5M solution in toluene) was added dropwise over 10 min. The reaction mixture was stirred for 1 h at 0-5° C. and then 2 h at RT. The light orange solution was cooled to 0-5° C., a solution of brine (2.5 mL) and 2M NaOH (2.5 mL) was added dropwise over 13 min. The reaction mixture was extracted with tert-butyl methyl ether (4.8 mL). The organic phase was separated and the aqueous solution was extracted with tert-butyl methyl ether (3.2 mL), the combined organic phases were dried (1.8 g of Na₂SO₄), filtered and evaporated in a rotary evaporator at 40° C. and 300-10 mbar. The residue was treated three times with a solution of tert-butyl methyl ether (0.2 mL) and toluene (1.2 mL) and evaporated at 40° C. and 300-10 mbar to afford 256 mg (82%) of (2-fluoro-6-hydroxymethyl-phenyl)-methanol (5) as an off white crystals: MS (EI): 138 (100), 137 (94), 109 (57).

### 3.1.a) Synthesis of 6-benzhydryl-3-trifluoromethyl-6,7-dihydro-5H-pyrrolo[3,4-b]pyridine

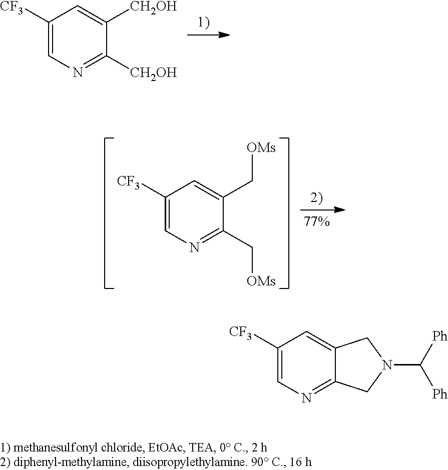

A 1.5 l four-necked round bottom flask equipped with a thermometer, a dropping funnel, an intensive cooler, a mechanical stirrer and an inert gas supply was charged with a solution of (2-hydroxymethyl-5-trifluoromethyl-pyridin-3-yl)-methanol (69 g, 333.1 mmol) and EtOAc (900 mL), cooled to 0° C. and treated over ca. 10 min at Tᵢ=0-5° C. with methanesulfonyl chloride (83.7 g, 730.7 mmol). The brown solution was stirred at Tᵢ=0-5° C. for 10 min and treated over 60 min at Tᵢ=0-5° C. with a solution of NEt₃ (85.1, 836.4 mmol) in 200 mL EtOAc (exothermic reaction). The yellow suspension was stirred for 2 h at Tᵢ=0-5° C. then filtered and the residue was washed with EtOAc (200 mL). The combined dimesylate filtrate was treated at Tᵢ=5-10° C. with diphenylmethyl amine (61.0 g, 333.0 mmol) and DIPEA (87.8 g, 666.0 mmol). The reaction mixture was heated to Tᵢ=76-80° C. and stirred at this temperature for 2.5 h and over night under reflux.

The mixture was poured in one portion into 940 mL of water (exothermic reaction), vigorously stirred for 10 min and then the phases separated. The organic phase was washed with half saturated NaCl solution (900 mL) and dried (Na₂SO₄). The solvent of the organic phase was evaporated (60° C., 300 to 10 mbar) to afford 115 g of crude 6-benzhydryl-3-trifluoromethyl-6,7-dihydro-5H-pyrrolo[3,4-b]pyridine as a beige residue containing traces of EtOAc.

The crude product was treated with iso-propanol (IPA) (500 mL) and stirred at 60° C. for 10 min. The solvent of the light brown suspension was evaporated again and the procedure repeated a second time. The residue was dissolved at 80° C. in IPA (1.65 L) and filtered over charcoal. The charcoal was rinsed with IPA (170 mL) and the total volume concentrated to 900 mL. The solution was treated at Tᵢ=75-80° C. with 300 mL of H₂O. The suspension was cooled down over 2 h to RT and stirred over night at RT. The thick light brown suspension was then stirred for 1 h at 40° C., cooled over 5 h to 0° C. and stirred at this temperature over night. The suspension was filtered, the crystals rinsed with IPA (340 mL) and dried at 50° C. and 10 mbar over two days to afford 73.5 g (62.3%) of 6-benzhydryl-3-trifluoromethyl-6,7-dihydro-5H-pyrrolo[3,4-b]pyridine as white crystals: MS (turbo spray): 355 (M+H⁺, 100), 167 (37).

### 3.1.b) Synthesis of 6-benzhydryl-3-trifluoromethyl-6,7-dihydro-5H-pyrrolo[3,4-b]pyridine hydrochloride salt

A 350 mL four-necked round bottom flask equipped with a thermometer, a dropping funnel, an intensive cooler, a mechanical stirrer and an inert gas supply was charged with methanol (117 mL) and treated over 5 min at Tᵢ=15-30° C. with acetyl chloride (2.6 mL, 1 36.3 mmol) (exothermic reaction). The colorless solution was stirred for 10 min at RT and treated in one portion with 11.7 g (33.0 mmol) of 6-benzhydryl-3-trifluoromethyl-6,7-dihydro-5H-pyrrolo[3,4-b]pyridine. The powder funnel was rinsed with MeOH (20 mL) and the clear yellow solution was stirred at RT for 15 min. The solvent was evaporated at 60° C. under reduced pressure to a total volume of 20 mL and tert-butyl-methyl ether (210 mL) was added at 60° C. The mixture was heated to reflux and stirred at this temperature for 40 min then cooled over 1 h to RT. The yellow suspension was filtered, the crystals rinsed with tert-butyl-methyl ether (22 mL) and dried at RT and <10 mbar for 12 h to afford 11.5 g (89.2%) of 6-benzhydryl-3-trifluoromethyl-6,7-dihydro-5H-pyrrolo[3,4-b]pyridine hydrochloride salt as light-yellow crystals: MS (EI): 355 (M+H⁺, 100), 167 (9).

### 3.2. Synthesis of 6,6-dibenzyl-3-trifluoromethyl-6,7-dihydro-5H-pyrrolo[3,4-b]pyridin-6-ium methanesulfonate

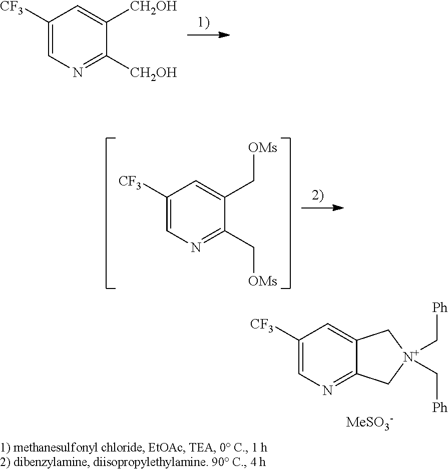

A 500 mL four-necked round bottom flask equipped with a thermometer dipping into the reaction mixture, a dropping funnel, an intensive condenser, a mechanical stirrer and an inert gas supply was charged with a solution of (2-hydroxymethyl-5-trifluoromethyl-pyridin-3-yl)-methanol, (10.36 g, 50.0 mmol) and EtOAc (180 mL). The solution was cooled to 0-5° C. and treated with methanesulfonyl chloride (8.5 mL, 110.0 mmol), Tᵢ rose from 1.0 to 2.0° C. The brown solution was stirred at Tᵢ=0-5° C. and treated over 70 min at Tᵢ=0-5° C. with a solution of TEA (17.4 mL, 125.0 mmol) in EtOAc (19 mL) (exothermic reaction). The yellow suspension was stirred for 0.5 h at Tᵢ=0-5° C. and filtered. The residue was washed with EtOAc (65 mL). The combined dimesylate filtrates were treated at Tᵢ=5-10° C. with dibenzylamine (9.6 mL, 50.0 mmol), DIPEA (9.4 mL, 55.0 mmol) and ethanol (13.2 mL). The reaction mixture was heated to reflux (Tᵢ=74° C.) and stirred at this temperature for 2 h. To the light brown suspension was added an additional 0.5 mL dibenzylamine (2.5 mmol) and refluxed for another 2.5 h to complete the reaction.

The light brown suspension was cooled to RT over 2 h, stirred for 16 h at RT, then cooled to 0-5° C. and stirred for 2 h at this temperature. The light brown suspension was filtered over a pre-cooled (0-5° C.) glass filter funnel G3. The filter cake was washed with a pre-cooled (0-5° C.) mixture of EtOAc (80 mL) and ethanol (5 mL). The white crystals were dried in a rotary evaporator at 40° C./3 mbar for 6 h to afford 16.2 g (69.6%) of 6,6-dibenzyl-3-trifluoromethyl-6,7-dihydro-5H-pyrrolo[3,4-b]pyridin-6-ium methanesulfonate as white crystals: MS (EI): 369 (M⁺, 100), 277 (8).

### 3.3.a) Preparation of Methanesulfonic acid 2-fluoro-6-methanesulfonyloxymethyl-benzyl ester

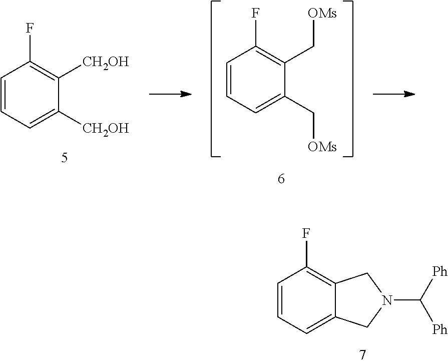

A 200 mL two necked round bottom flask equipped with a thermometer, a magnetic stirrer and an inert gas supply was charged with a solution of 5 (10.15 g, 65.0 mmol) and EtOAc (208 mL), cooled to 0-5° C., and, methanesulfonyl chloride (11.1 mL, 143.0 mmol) was added dropwise over 5 min. A solution of TEA (22.6 mL, 162.5 mmol) and EtOAc (25 mL) was added dropwise over 38 min. The white suspension was stirred for 2 h at 0-5° C., filtered over a pre-cooled (0-5° C.) glass filter funnel and washed with pre-cooled (0-5° C.) EtOAc (65 mL) to afford 325 mL of a solution containing 6.

### 3.3.b) Preparation of 2-Benzhydryl-4-fluoro-2,3-dihydro-1H-isoindole

A 500 mL two necked round bottom flask equipped with a thermometer a reflux condenser a magnetic stirrer and an inert gas supply was charged with a solution of 6 and EtOAc (325 mL, 65 mmol) from the previous step, then aminodiphenylmethane (12.3 mL, 71.5 mmol) and DIPEA (27.8 mL 162.5 mmol) were added. The reaction mixture was heated under reflux for 6 h, cooled to RT and the solvent was evaporated at 40° C. at 160 to 10 mbar to afford 49 g of crude 2-benzhydryl-4-fluoro-2,3-dihydro-1H-isoindol as a light pink solid. The crude product was treated with methanol (197 mL) and heated to reflux for 15 min. The light brown suspension was cooled to RT over 30 min stirred for 2 h at RT and cooled to 0-5° C. and stirred for 30 min. The suspension was filtered over a pre-cooled glass filter funnel and the residue was washed with cold methanol (66 mL). The white crystals were dried at 40° C. and 10 mbar to afford 32.4 g (83%) of 2-benzhydryl-4-fluoro-2,3-dihydro-1H-isoindole (7): MS (turbo spray) 304 (M+H⁺, 39), 346 (11), 167 (100).

### 3.3.c) Preparation of 4-fluoro-2,3-dihydro-1H-isoindol hydrochloride (8)

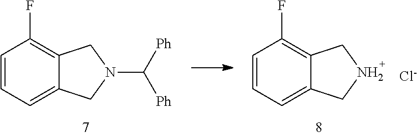

A 250 mL four necked round bottom flask equipped with a thermometer a magnetic stirrer and an hydrogen gas supply was charged with methanol (35 mL) cooled to 0-5° C. and acetyl chloride (1.80 mL, 25.3 mmol) was slowly added. The temperature rose to 20° C. and this mixture was stirred for 15 min at RT, before adding a solution of 7 (6.98 g, 23.0 mmol) and methanol (35 mL). The white suspension was stirred for 10 min at RT and 10% Pd/C (0.70 g) was added. The vessel was evacuated three times and ventilated, first with argon, then with hydrogen. The black mixture was hydrogenated for 7 h at RT, filtered and the filter cake was washed with methanol (20 mL). The light yellow filtrate was treated again with 10% Pd/C (0.70 g) and hydrogenated for another 15 h at RT. The black suspension was filtered and the filter cake was washed with methanol (25 mL). The clear colorless filtrate was evaporated at 40° C. and 300 to 10 mbar to afford 7.34 g of a white residue. The crude product was suspended in tert-butyl methyl ether (40 mL), heated to refluxed for 10 min, cooled to RT and stirred for 3 h at RT. The suspension was filtered and the filter cake was washed with tert-butyl methyl ether (13 mL). The white crystals were dried at 40° C. and 10 mbar to afford 3.74 g (94%) of 4-fluoro-2,3-dihydro-1H-isoindole hydrochloride as white crystals: MS (turbo spray) 138 (M+H⁺, 100), 139 (10).

### 3.4.a) same reaction as in 3.3.a)

### 3.4.b) Preparation of 2,2-dibenzyl-4-fluoro-2,3-dihydro-1H-isoindolium methanesulfonate (9)

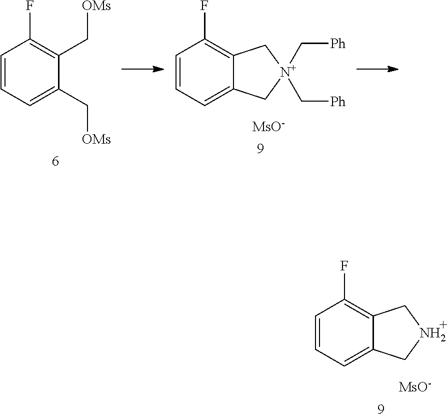

A 50 mL two necked round bottom flask equipped with a thermometer a magnetic stirrer a reflux condenser and an inert gas supply was charged with 9 (1.56 g, 5.0 mmol) EtOAc (24.0 mL) and maintained under argon. The clear yellow solution was treated with ethanol (1.3 mL), dibenzylamine (0.96 mL, 5.0 mmol) and DIPEA (0.94 mL, 5.5 mmol). The reaction mixture was heated to reflux for 2.5 h, then dibenzylamine (0.05 mL, 0.25 mmol) and DIPEA (0.04 mL, 0.25 mmol) were added and the mixture was refluxed for another 1.5 h. The clear yellow solution was cooled to RT and the solvent evaporated at 40° C. and 200 to 5 mbar to afford 3.48 g crude product as a yellow oil. The crude product was purified by SiO₂ chromatography (85 g SiO₂) eluted with a mixture of CH₂Cl₂, methanol, 25% aqueous ammonium hydroxide (90/10/1). The combined fractions were evaporated and dried on a rotary evaporator at 40° C. and 250 to 10 mbar to afford 1.14 g (55%) of 9 as a white solid: MS (turbo spray) 318 (M⁺, 100), 319 (24), 226 (7)

### 3.4.c) Preparation of 4-fluoro-2,3-dihydro-1H-isoindol methanesulfonic acid (10)

A 10 mL two necked round bottom flask equipped with a thermometer a magnetic stirrer, and a hydrogen gas supply was charged with 9 (827 mg, 2.0 mmol), methanol (4.1 mL) and 10% Pd/C (41.0 mg). The vessel was evacuated three times and ventilated, first with argon, then with hydrogen. The black mixture was hydrogenated for 2.5 h at RT, filtered and the filter cake was washed with methanol (6.0 mL). The clear filtrate was evaporated at 40° C. and 300 to 10 mbar yielding 451 mg (97%) of 10 as a grey solid: MS (turbo spray) 138 (M+H⁺, 100), 139 (6).

### 4.1. Synthesis of 3-trifluoromethyl-6,7-dihydro-5H-pyrrolo[3,4-b]pyridin-6-ium chloride

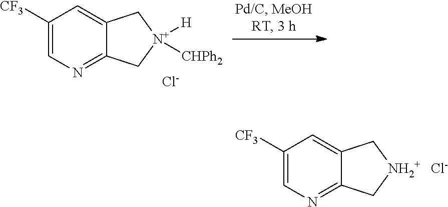

An autoclave was charged with a suspension of 6-benzhydryl-3-trifluoromethyl-6,7-dihydro-5H-pyrrolo[3,4-b]pyridine hydrochloride salt (150 g, 383.8 mmol), Pd/C (7.5 g) and methanol (0.5 L). The suspension was stirred at RT. The autoclave was sealed, the hydrogen pressure set at 1.5 bar (absolute) and the hydrogenation was conducted with stirring at 20-25° C. for 3-4 h. The autoclave was vented, the suspension filtered and the black residue was rinsed twice with methanol (0.5 L, total of 1 L). The filtrate was evaporated at 60° C. under reduced pressure to yield 152.3 g of crude 3-trifluoromethyl-6,7-dihydro-5H-pyrrolo[3,4-b]pyridin-6-ium chloride as brown residue.

The clear yellow solution of 152.3 g crude product 3-trifluoromethyl-6,7-dihydro-5H-pyrrolo[3,4-b]pyridin-6-ium chloride in methanol (200 mL) was diluted with 600 mL EtOAc and the solvent was concentrated at 60° C. under reduced pressure (335-250 mbar) to a total volume of 500 mL. Methanol was exchanged at 60° C., keeping the volume constant under reduced pressure by the addition of 3.5 l EtOAc. The resulting suspension was treated at 60° C. with toluene (125 mL), stirred for 15 min and methanol (50 mL) was added. After stirring for 15 min at 60° C. the suspension was cooled over 1 h to RT and stirred for 1 h. The crystals were filtered and rinsed with a mixture of EtOAc (300 mL), toluene (10 mL) and methanol (10 mL) then dried at 60° C. and <10 mbar for 2 h and overnight at RT to afford 74.8 g (86.7%) of 3-trifluoromethyl-6,7-dihydro-5H-pyrrolo[3,4-b]pyridin-6-ium chloride as a beige powder: MS (EI): 189 (M+H⁺, 100).

### 4.2. Synthesis of 3-trifluoromethyl-6,7-dihydro-5H-pyrrolo[3,4-b]pyridin-6-ium methanesulfonate

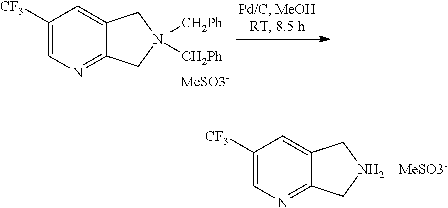

A 200 mL four-necked round bottom flask equipped with a thermometer probe dipping into reaction mixture, a mechanical hydrogenation stirrer, an inert gas supply and a hydrogenation supply apparatus was charged with a solution of 6,6-dibenzyl-3-trifluoromethyl-6,7-dihydro-5H-pyrrolo[3,4-b]pyridin-6-ium methanesulfonate (15.8 g, 34.0 mmol) and Pd/C (0.39 g) in methanol (79 mL) which was then stirred at RT. The reaction flask was ventilated several times with argon and hydrogen. The hydrogenation was performed under atmospheric pressure and stirred at 20-25° C. for 8.5 h. The reaction flask was ventilated several times with argon and the suspension was filtered. The black residue was rinsed twice with methanol (13 mL, a total of 26 mL).

From the clear light brown filtrate, 80 mL methanol was distilled off (80° C./480-280 mbar), then the remaining methanol was exchanged at Tᵢ 38-43° C. and 560-370 mbar, keeping the volume constant by twice adding EtOAc (76 mL, a total of 152 mL). Crystallization commenced and a further 76 mL EtOAc were added. The light brown suspension was heated at reflux for 5 min, cooled to RT within 0.5 h and stirred for 1 h at RT. The suspension was filtered and the off-white crystals were rinsed in portions with a total of 25 mL EtOAc. The crystals were dried in a rotary evaporator at 42° C. and <10 mbar for 6 h to afford 8.87 g (91.8%) of 3-trifluoromethyl-6,7-dihydro-5H-pyrrolo[3,4-b]pyridin-6-ium methanesulfonate as an off-white powder: MS (EI): 189 (M+H⁺, 100).

### 5. Synthesis of (S)-trifluoro-isopropanol as described in PCT/EP2007057372 filed on Jul. 7, 2007

A 185-mL stirred autoclave equipped with an electric heating/cooling system, temperature cascade control, measurement of temperature, of gas flow, of stirring rate and of pressure was connected to a hydrogen feed system. During the operation the temperature, the stirring rate, the hydrogen flow rate as well as the hydrogen consumption were recorded on-line through a PC data gathering system.

In the glove box the 185-mL autoclave was charged with water (0.75 mL) and stored in the refrigerator (−18° C.) for 2 h. The autoclave was removed from the refrigerator and charged with RuCl₂((S)-3,5-tent-Bu-MeOBIPHEP)((R,R)-DPEN (14.86 mg 10.5×10⁻³ mmol, substrate-to-catalyst ratio of 20'000), sodium formate (75.00 mg, 1.10 mmol) and cold (−18° C.) trifluoroacetone (23.44 g, 18.75 mL, 209.2 mmol), sealed immediately and pressurized with 7 bar of argon. The autoclave was removed from the glove box and introduced into a heating/cooling unit and connected to a hydrogenation line which was thoroughly flushed with hydrogen. The autoclave was pressurized with 40 bar of hydrogen under stirring and the heating started. After 10 h, the reaction temperature was increased to 60° C. for 2 h to complete the conversion. Then the autoclave was cooled and vented and the crude reaction mixture transferred into a 30 mL round-bottomed flask to afford 24.55 g of crude (S)-trifluoro-isopropanol as an off-white suspension. The crude product was distilled at 34° C./150 mbar to afford 23.91 g (96.3%) of (S)-trifluoro-isopropanol as a colorless oil: H₂O content 3.4%; cf. EA; Ru-content<1 ppm, not detected.

### 6. Synthesis of 5-methanesulfonyl-2-(2,2,2-trifluoro-1-methyl-ethoxy)-benzoic acid as described in EP application no. 07103485.4

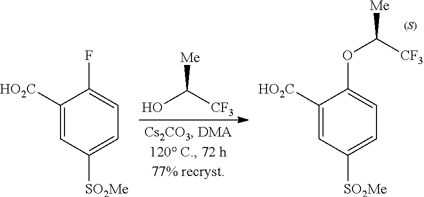

A 12 L autoclave was charged with a colorless solution of 2-fluoro-5-methanesulfonyl-benzoic acid (700.0 g, 3.2 mol) in dimethylacetamide (7.7 L). The solution was treated with cesium carbonate (1965.0 g, 6.0 mol) and (S)-trifluoro-isopropanol (522.8 g, 4.6 mol). The white reaction suspension was warmed to 120° C. and stirred under argon for 72 h (1.5 bar). After cooling to 20° C., the white suspension was filtered, the filter cake was washed with dimethylacetamide (500 mL) and the filtrate was evaporated. To the residue was added water (9 L) and the solution was extracted 3 times with EtOAc (7 L, or a total of 21 L). The aqueous phase was heated in the rotary evaporator to completely remove residual EtOAc from the water phase. The pH of the water phase was adjusted to 1.5 by addition of 37% HCl (600 mL) whereby the product precipitated. The suspension was stirred at RT for 1 h, filtered, the crystals were washed with water (5 L) and dried under high vacuum for 24 h at 50° C. to yield 840.0 g (84.0%) of 5-methanesulfonyl-2-(2,2,2-trifluoro-1-methyl-ethoxy)-benzoic acid as white crystals.

### 7.1. Synthesis of [5-methanesulfonyl-2-(2,2,2-trifluoro-1-methyl-ethoxy)-phenyl]-(3-trifluoromethyl-6,7-dihydro-5H-[1]pyridin-6-yl)-methanone

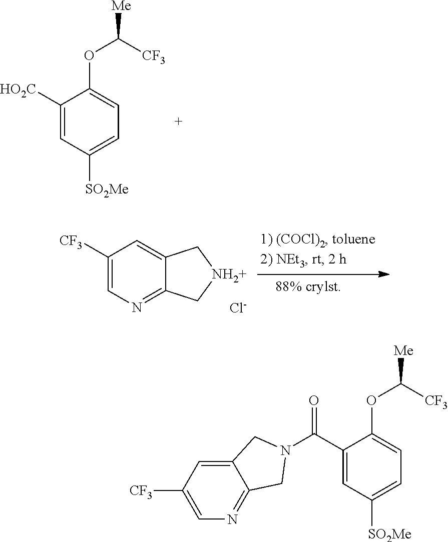

A 250 mL four-necked round bottom flask equipped with a thermometer, a dropping funnel, a mechanical stirrer and an inert gas supply was charged with 5-methanesulfonyl-2-(2,2,2-trifluoro-1-methyl-ethoxy)-benzoic acid (44.9 g, 143.7 mmol), DMF (1.8 mL) and toluene (295 mL) and a solution of oxalyl chloride (18.3 g, 141.3 mmol) and toluene (32 mL) was added over 14 min at RT (exothermic reaction) The suspension was stirred for 1 h at RT and added dropwise at RT over 16 min into a 2.5 l four-necked round bottom flask charged with a solution of 3-trifluoromethyl-6,7-dihydro-5H-pyrrolo[3,4-b]pyridine (32.0 g) of example 4.1. in toluene (900 mL) and TEA (61.3 g, 605.6 mmol). The reaction mixture was stirred at RT for 2.5 h then the suspension was filtered and the residue washed in portions with toluene (190 mL). The filtrate was extracted with water (860 mL). After separation of the phases, the organic phase was washed three times with 5% NaHCO3 solution (590 mL, a total of 1.8 L) and once with 5% NaCl solution (600 mL). The organic phase was dried (Na₂SO₄), filtered and the solvent of the organic phase was evaporated at 60° C. and 400 mbar. The residue was treated with ethanol (400 mL) and the solvent again evaporated at 60° C. under reduced pressure to yield crude 70.7 g of [5-methanesulfonyl-2-(2,2,2-trifluoro-1-methyl-ethoxy)-phenyl]-(3-trifluoromethyl-6,7-dihydro-5H-[1]pyridin-6-yl)-methanone as a light yellow foam.

Crystallization from ethanol/water: A yellow solution of 70.7 g [5-methanesulfonyl-2-(2,2,2-trifluoro-1-methyl-ethoxy)-phenyl]-(3-trifluoromethyl-6,7-dihydro-5H-[1]pyridin-6-yl)-methanone in 194 mL ethanol was heated at reflux and treated over 30 min with 194 mL water. The mixture was cooled over 1 h to Tᵢ=38-42° C. and stirred for 1 h at this temperature (seeding at Tᵢ=53-55° C.). To the white thick suspension was added at 47 min water (580 mL) and the suspension was cooled within 30 min to RT and stirred for 1 h. The white suspension was filtered, the crystals were rinsed with a mixture of ethanol (32 mL) and water (128 mL), then dried at 50° C. and 5 mbar for 15 h to yield 62.7 g of [5-methanesulfonyl-2-(2,2,2-trifluoro-1-methyl-ethoxy)-phenyl]-(3-trifluoromethyl-6,7-dihydro-5H-[1]pyridin-6-yl)-methanone as a light yellow powder.

Crystallization from ethanol/heptane: A yellow solution of 62.7 g [5-methanesulfonyl-2-(2,2,2-trifluoro-1-methyl-ethoxy)-phenyl]-(3-trifluoromethyl-6,7-dihydro-5H-[1]pyridin-6-yl)-methanone in ethanol (150 mL) was heated at reflux, stirred for 10 min, then cooled over 1 h to Tᵢ=38-42° C. and stirred for 1 h at this temperature (seeding with form A at Tᵢ=53-55° C.). The thick suspension was cooled over 60 min to RT and treated over 60 min with 450 mL heptane. After stirring for 1 h, the white suspension was filtered and the crystals were rinsed with a mixture of ethanol (34 mL) and heptane (66 mL) then dried at 50° C. and <10 mbar over night to yield 58.8 g (88.0%) of [5-methanesulfonyl-2-(2,2,2-trifluoro-1-methyl-ethoxy)-phenyl]-(3-trifluoromethyl-6,7-dihydro-5H-[1]pyridin-6-yl)-methanone as white crystals: MS (EI): 505 (M+Na⁺, 44), 483 (M+H⁺, 100), m.p.: 147.3° C.

### 7.2. Synthesis of [5-methanesulfonyl-2-(2,2,2-trifluoro-1-methyl-ethoxy)-phenyl]-(3-trifluoromethyl-6,7-dihydro-5H-[1]pyridin-6-yl)-methanone

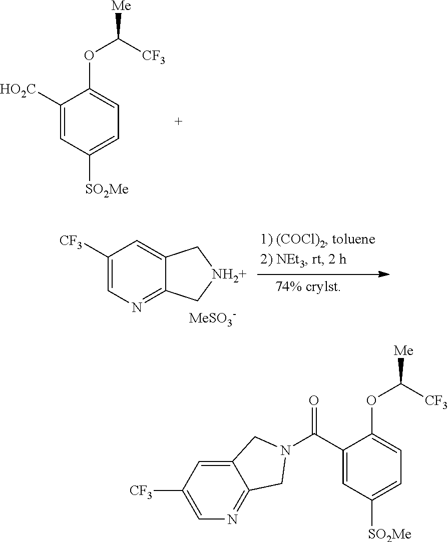

A 100 mL four-necked round bottom flask equipped with a thermometer, a dropping funnel, a mechanical stirrer and an inert gas supply was charged with an off-white suspension of 5-methanesulfonyl-2-(2,2,2-trifluoro-1-methyl-ethoxy)-benzoic acid (3.12 g, 10.0 mmol), DMF (0.12 mL) and toluene (39.0 mL). The suspension was treated at RT over 10 min with a solution of oxalyl chloride (0.86 mL, 10.0 mmol) and toluene (2.2 mL) (exothermic reaction, gas evolution). The turbid solution was stirred for 1.5 h at RT then added to a 250 mL four-necked round bottom flask equipped as mentioned above charged with a solution of 2.84 g 3-trifluoromethyl-6,7-dihydro-5H-pyrrolo[3,4-b]pyridin-6-ium methanesulfonate (10.0 mmol) from example 4.2. in EtOAc (84.0 mL) and TEA (5.85 mL, 42.0 mmol) at RT over 15 min. The reaction mixture was stirred at RT for 2.5 h.

The light brown suspension was extracted with 1M aqueous HCl (80.0 mL). The organic phase was washed three times 1M NaHCO₃ (50 mL, a total of 150 mL) and once with 12% NaCl solution. The combined organic phase was dried (Na₂SO₄, 110 g), filtered over a glass fiber filter and the residue was washed in portions with EtOAc (60 mL total). The combined filtrates were evaporated (42° C. and 400-5 mbar) and the residual off-white foam was treated with ethanol (20 mL). The solvent was removed at 42° C. and 150-5 mbar to afford 4.47 g of crude [5-methanesulfonyl-2-(2,2,2-trifluoro-1-methyl-ethoxy)-phenyl]-(3-trifluoromethyl-6,7-dihydro-5H-[1]pyridin-6-yl)-methanone as off-white foam

Crystallization from ethanol/water: A yellow-brown solution of 4.45 g crude product in ethanol (13.4 mL) was heated to 61° C. and treated over 5 min with water (13.4 mL). The mixture was cooled over 1 h to Tᵢ=38-42° C. and stirred for 1 h at this temperature then water (39.5 mL) was added to the white thick suspension over 105 min and the suspension was cooled over 60 min to RT and stirred for 16 h. The white suspension was filtered, the crystals were rinsed with a mixture of ethanol (1.9 mL) and water (7.6 mL) and dried at 40° C. and <10 mbar for 6 h to afford 4.03 g of [5-methanesulfonyl-2-(2,2,2-trifluoro-1-methyl-ethoxy)-phenyl]-(3-trifluoromethyl-6,7-dihydro-5H-[1]pyridin-6-yl)-methanone as a off-white powder

Crystallization from ethanol/heptane: A yellow-brown solution of [5-methanesulfonyl-2-(2,2,2-trifluoro-1-methyl-ethoxy)-phenyl]-(3-trifluoromethyl-6,7-dihydro-5H-[1]pyridin-6-yl)-methanone (4.03 g) in ethanol (9.3 mL) was heated at reflux, stirred for 3 min, cooled over 35 min to Tᵢ=38-42° C. and stirred for 2.5 h at this temperature resulting in an off-white suspension. This suspension was cooled over 60 min to RT, stirred for 16 h, treated dropwise over 16 min with heptane (27 mL) and stirred at RT again for 6 h. The white suspension was filtered, the crystals were washed with a mixture of ethanol (1.5 mL) and heptane (4.5 mL) then dried over night at 40° C. and <10 mbar to afford 3.55 g (74%) of [5-methanesulfonyl-2-(2,2,2-trifluoro-1-methyl-ethoxy)-phenyl]-(3-trifluoromethyl-6,7-dihydro-5H-[1]pyridin-6-yl)-methanone as white crystals: MS (EI): 505 (M+Na⁺, 32), 483 (M+H⁺, 100).

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A process of preparing a compound of formula II or a salt thereof 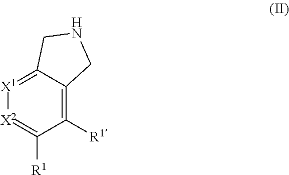
wherein
X¹ is N or C;
X² is C when X¹ is N, or is C or N when X¹ is C;
R¹ and R^{1′} are each independently hydrogen, hydroxy, halogen, lower alkyl optionally substituted by halogen or hydroxy, lower alkoxy optionally substituted by halogen, cycloalkyl, —CN, —NH₂, —S-lower alkyl, —S(O)₂-lower alkyl, —O—(CH₂)_{y}-lower alkoxy, —O(CH₂)_{y}C(O)N(lower alkyl)₂, —C(O)-lower alkyl, —C(O)O-lower alkyl, —C(O)—NH-lower alkyl, or —C(O)—N(lower alkyl)₂;
y is 1, 2, 3 or 4;
comprising:
a) carbonylating compound (1) 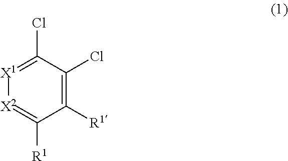
in the presence of CO, alcohol, and a PdCl₂ (dppf) catalyst, wherein dppf is 1,1′-bis(diphenylphosphino)ferrocene, to obtain diester (2) 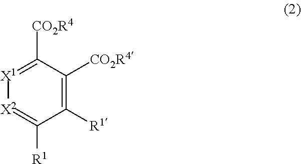
wherein R⁴ and R^{4′} are each independently C₁-C₆ alkyl;
b) reducing diester (2) in the presence of a reducing agent selected from the group consisting of NaBH₄ in acetic acid, diisobutylaluminum hydride, LiBH₄ and LiAlH₄ and optionally in the presence of an alkali and/or alkaline earth salt or transition metal salt to obtain diol (3) 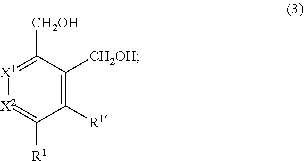
c) forming a bis-sulfonate (4) 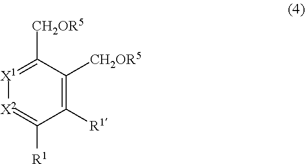
wherein R⁵ and R^{5′} are each independently a sulfonate leaving group selected from the group consisting of nosylate, tosylate and mesylate; in presence of a base selected from the group consisting of diisopropylethylamine (DIPEA) and triethylamine (NEt₃), in an EtOAc solvent, wherein insoluble salts are optionally formed and easily removed by filtration;
d) cyclizing the bis-sulfonate (4) with a protected secondary amine, HNR⁶R^{6′}, to obtain a protected amine (5) or its salt 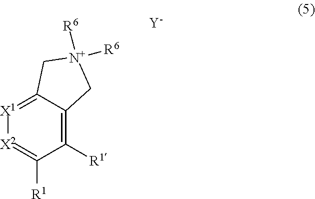
wherein R⁶ and R^{6′} are each independently hydrogen or an amino protecting group susceptible to hydrogenolytic cleavage and Y⁻ is an anionic gegenion, at a temperature of 60° C. to 100° C. in a solvent selected from the group consisting of THF, MeOH, or DMF in the presence of a base selected from the group consisting of DIPEA, K₂CO₃ and NEt₃; and
e) hydrogenolyzing the protected amine (5), or its salt, with hydrogen in the presence of catalytic amounts of Pd/C, optionally in the presence of an acid to afford the compound of formula II or a salt thereof.

2. A process of preparing a compound of formula I: 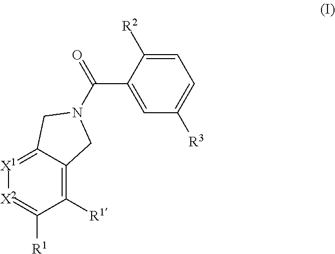
wherein:
X¹ is N or C;
X² is C when X¹ is N, or is C or N when X¹ is C;
R¹ and R^{1′} are independently of each other hydrogen, hydroxy, halogen, lower alkyl optionally substituted by halogen or hydroxy, lower alkoxy optionally substituted by halogen, cycloalkyl, —CN, —NH₂, —S-lower alkyl, —S(O)₂-lower alkyl, —O—(CH₂)_{y}-lower alkoxy, —O(CH₂)_{y}C(O)N(lower alkyl)₂, —C(O)-lower alkyl, —C(O)O-lower alkyl, —C(O)—NH-lower alkyl, or —C(O)—N(lower alkyl)₂;
y is 1, 2, 3 or 4;
R² is halogen, lower alkyl optionally substituted by halogen or hydroxy, lower alkoxy optionally substituted by halogen, cycloalkyl, —NR⁷R^{7′}, cyclic amine, heterocycloalkyl, aryl or 5- or 6-membered heteroaryl, containing one, two or three heteroatoms, selected from the group consisting of oxygen, sulphur or nitrogen;
R³ is —S(O)₂-lower alkyl, —S(O)₂NH-lower alkyl, —NO₂ or —CN;
R⁷ and R^{7′} are independently of each other hydrogen or lower alkyl;
comprising coupling a compound of formula II, or a salt thereof, 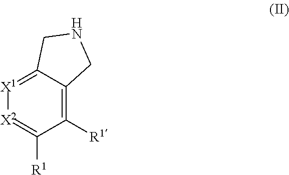
with a compound (7) 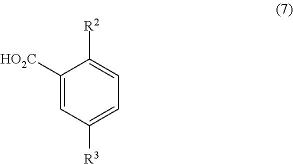
in the presence of a of an activating agent selected from the group consisting of POCl₃, (COCl)₂, and SOCl₂ and a coupling reagent selected from the group consisting of 2-(1H-7-azabenzotriazol-1-yl)-1,1,3,3-tetramethyl uronium hexafluorophosphate methanaminium (HATU), O-(benzotriazol-1-yl)-N,N,N′,N′-tetramethyluronium tetrafluoroborate (TBTU), and N,N′-carbonyldiimidazole (CDI);
wherein compound (II) is prepared by the process comprising
a) carbonylating compound (1) 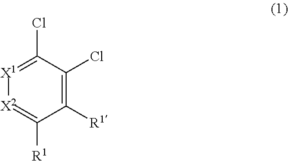
in the presence of CO, alcohol, and a catalyst selected from the group consisting of PdCl₂ (dppf), wherein dppf is 1,1′-bis(diphenylphosphino)ferrocene, to obtain diester (2) 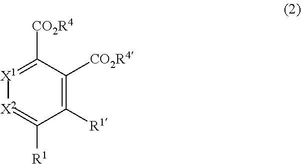
wherein R⁴ and R^{4′} are each independently C₁-C₆ alkyl;
b) reducing diester (2) in the presence of a reducing agent selected from the group consisting of NaBH₄ in acetic acid, diisobutylaluminum hydride, LiBH₄ and LiAlH₄ and optionally in the presence of an alkali and/or alkaline earth salt or transition metal salt to obtain diol (3) 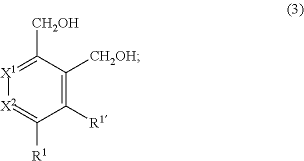
c) forming a bis-sulfonate (4) 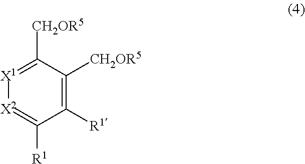
wherein R⁵ and R^{5′} are each independently a sulfonate leaving group selected from the group consisting of nosylate, tosylate and mesylate; in presence of a base selected from the group consisting of diisopropylethylamine (DIPEA) and triethylamine (NEt₃), in an EtOAc solvent, wherein insoluble salts are optionally formed and easily removed by filtration;
d) cyclizing the bis-sulfonate (4) with a protected secondary amine, HNR⁶R^{6′}, to obtain a protected amine (5) or its salt 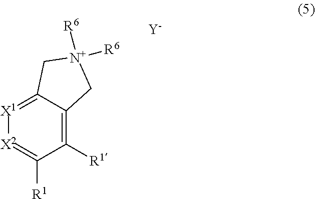
wherein R⁶ and R^{6′} are each independently hydrogen or an amino protecting group susceptible to hydrogenolytic cleavage and Y⁻ is an anionic gegenion, at a temperature of 60° C. to 100° C. in a solvent selected from the group consisting of THF, MeOH, or DMF in the presence of a base selected from the group consisting of DIPEA, K₂CO₃ and NEt₃; and
e) hydrogenolyzing the protected amine (5), or its salt, with hydrogen in the presence of catalytic amounts of Pd/C, optionally in the presence of an acid to afford the compound of formula II or a salt thereof.

3. A process of claim 2 wherein R¹ is alkyl substituted by halogen, R¹′ is hydrogen, X¹ is N, X² is CH, R² is —O—CH(CH₃)CF₃ and R³ is —S(O)₂-lower alkyl.
